SEQUENCE LISTING (3 sequences)

SEQ ID NO: 1 - DNA, 20 bp, Mus musculus
  source 1..20: mol_type = genomic DNA
cacggataacagcgccctgt

SEQ ID NO: 2 - DNA, 24 bp, synthetic construct
  source 1..24: mol_type = other DNA
cacgtttgtacaagccagaactta

SEQ ID NO: 3 - DNA, 21 bp, synthetic construct
  source 1..21: mol_type = other DNA
tcttttggtcttcccgagcct

(19) United States
(12) Patent Application Publication
(10) Pub. No.: US 2026/0144238 A1
(43) Pub. Date: May 28, 2026

(54) SKIN TISSUE

(71) Applicant: The University of Tokyo, Tokyo (JP)

(72) Inventors: Naoaki MIZUNO, Tokyo (JP); Hiromitsu NAKAUCHI, Tokyo (JP); Hisato NAGANO, Tokyo (JP); Hideki MASAKI, Tokyo (JP); Motoo WATANABE, Tokyo (JP); Hideyuki SATO, Tokyo (JP)

(21) Appl. No.: 18/707,335

(22) PCT Filed: Nov. 4, 2022
(86) PCT No.: PCT/JP2022/041189

§ 371 Date: May 3, 2024

(30) Foreign Application Priority Data
Nov. 5, 2021 (JP) 2021-180974

(51) Int. Cl. A01K 67/0271; A01K 67/0276; C12N 5/071; C12N 5/074

(52) U.S. Cl. CPC A01K 67/0271; A01K 67/0276; C12N 5/0625; C12N 5/0696; A01K 2207/12; A01K 2217/075; A01K 2227/10; A01K 2267/025

(57) ABSTRACT

The present disclosure provides an isolated skin tissue comprising an epidermis, a basement membrane, and a dermis, wherein the epidermis is attached to one surface of the basement membrane, and the dermis is attached to the other surface of the basement membrane; and the epidermis comprises first cells, and the dermis comprises second cells, wherein the second cells have no ability to differentiate into mature epidermal cells.

### SEQUENCE LISTING

The instant application contains a Sequence Listing which has been submitted electronically in XML format and is hereby incorporated by reference in its entirety. Said XML copy, created on Jan. 19, 2026, is named P1TD190280US Sequence listing and is 3,794 bytes in size.

### TECHNICAL FIELD

The present disclosure relates to a skin tissue. In a nonlimiting example, the present disclosure relates to an isolated skin tissue comprising an epidermis, a basement membrane, and a dermis, wherein the epidermis is attached to one surface of the basement membrane, and the dermis is attached to the other surface of the basement membrane; and the epidermis comprises first cells, and the dermis comprises second cells, wherein the second cells have no ability to differentiate into mature epidermal cells.

### BACKGROUND ART

Wounds or burns may cause skin damages extending into the dermis. Such damages cannot be autonomously repaired and therefore require the grafting of skin tissues. In the skin grafting, autografting is performed, and the skin obtained from another body surface of the body is grafted to a lost part of the skin. In this case, graft survival rates are very high. However, there exist cases where autografting is difficult due to a widespread skin damage. Furthermore, there are requests for avoiding autografting because autografting, even if possible, results in scar at a site where the skin has been collected.

Allogeneic or xenogeneic skin grafting methods cannot expect permanent engraftment due to immune rejection and the like in many cases. Attempts have been made to prepare cultured skin sheets from cells collected from patients themselves (Non Patent Literatures 1 to 6). However, the sheets obtained by these methods are generally sheets that lack the basement membrane and the dermis and contain only epidermal cells (i.e., cultured epidermal sheets). The cultured epidermal sheet thus obtained, even when grafted to a body surface, is not easy to engraft on the body surface due to a lack of the dermis. Another attempt has been made to prepare artificial skin substitutes in vitro and however, has failed to construct functionally thick dermal layers.

### CITATION LIST

### Non Patent Literature

Non Patent Literature 1: Rheinwald J G, Green H. Cell. 1975; 6: 331-343
Non Patent Literature 2: Matsumura H et al., Burns. 2016; 42: 769-76
Non Patent Literature 3: Hefton J M et al., Lancet. 1983; 2: 428-430
Non Patent Literature 4: Katz A B, Taichman L B. J Invest Dermatol. 1994; 102: 55-60
Non Patent Literature 5: Sakamoto M et al., Ann Plast Surg. 2017; 78 (6): 651-658
Non Patent Literature 6: Sakamoto M et al. PLoS One. 2020; 15 (8): e0237985

### SUMMARY OF INVENTION

The present disclosure provides a skin tissue. In a nonlimiting example, the present disclosure may provide an isolated skin tissue comprising an epidermis, a basement membrane, and a dermis, wherein the epidermis is attached to one surface of the basement membrane, and the dermis is attached to the other surface of the basement membrane; and the epidermis comprises first cells, and the dermis comprises second cells, wherein the second cells have no ability to differentiate into mature epidermal cells.

According to the present disclosure, a skin tissue comprising an epidermis, a basement membrane, and a dermis, wherein the epidermis is derived from donor cells, and the dermis is derived from the donor cells and host cells, has been obtained by administering donor cells having ability to differentiate into a mature epidermis to a host embryo having no ability to differentiate into mature epidermal cells. The present disclosure is based on such findings.

The present disclosure provides the following embodiments of the invention.

[1] An isolated skin tissue comprising an epidermis, a basement membrane (or a basal lamina), and a dermis, wherein the epidermis is attached to one surface of the basement membrane (or the basal lamina), and the dermis is attached to the other surface of the basement membrane (or the basal lamina); and
the epidermis comprises first cells, or preferably, the epidermis consist of first cells, and the dermis comprises second cells and optionally further comprise the first cells, wherein
the second cells have low or no ability to differentiate into mature epidermal cells.

[2] The skin tissue according to [1], wherein second individual's cells are cells having loss of function in p63.

[3] The skin tissue according to [1] or [2], wherein the epidermis is a mature epidermis.

[4] The skin tissue according to any of [1] to [3], wherein 95% or more of cells of the epidermis (preferably cells of the epidermis including basal cells) are the first cells, and 75% or more of cells of the dermis are the second cells.

[5]A method for preparing a skin tissue, comprising:
providing a host embryo incapable of (autonomously) developing a mature epidermis (or a host embryo thus engineered);
injecting donor cells having ability to differentiate into a mature epidermis to the host embryo, wherein the injection is performed under conditions suitable for engraftment of the cells to the skin;
allowing the obtained embryo to grow in the uterus of a pseudo-pregnant surrogate mother mammal by transferring to the uterus thereof to obtain an individual having a mature epidermis from the mammal; and
isolating a skin tissue comprising a mature epidermis comprising cells derived from the donor cells, a basement membrane, and a dermis comprising cells derived from the host from the obtained individual.

[6] The method according to [5], wherein the host embryo incapable of developing a mature epidermis is a p63-knockout embryo.

[7] The method according to [5] or [6], wherein the donor cells having ability to differentiate into a mature epidermis are cells selected from the group consisting of pluripotent cells and cells destined for the epidermis.

[8] The method according to any of [5] to [7], wherein the embryo is an embryo at any stage of development from an eight-cell stage to a blastocyst stage.

[9] The method according to any of [5] to [7], wherein the embryo is an embryo at any stage of development from a blastocyst stage to the formation of an epidermis comprising K8/K18-positive cells.

[10] The method according to any of [5] to [7], wherein the embryo is an embryo having an epidermis of K8/K18-positive cells.

[21] The skin tissue according to any of [1] to [4] or the method according to any of [5] to [7], wherein the second cells have no ability to differentiate into any or all mature epidermal cells selected from the group consisting of spinous cells, granular cells, and cornified cells.

[22] The skin tissue according to any of [1] to [4] or the method according to any of [5] to [7], wherein the second cells have no ability to differentiate into all mature epidermal cells selected from the group consisting of spinous cells, granular cells, and cornified cells.

[23] The skin tissue or the method according to [21], wherein the second cells have ability to differentiate into basal cells.

[24] The skin tissue or the method according to [22], wherein the second cells have ability to differentiate into basal cells.

[25] The skin tissue according to any of [1] to [4], the method according to any of [5] to [7], or the skin tissue or the method according to any of [21] to [24], wherein the first cells have p63 gene having a normal function.

[26] The skin tissue according to any of the above items, wherein 95% or more of epidermal cells are the first cells, and less than 5% of the epidermal cells are the second cells.

[27] The skin tissue according to any of the above items, wherein 70% or more of dermal cells are the second cells, and less than 30% of the dermal cells are the first cells.

[28] The skin tissue according to any of the above items, wherein 95% or more of epidermal cells are the first cells, and less than 5% of the epidermal cells are the second cells; and 70% or more of dermal cells are the second cells, and less than 30% of the dermal cells are the first cells.

[29] The skin tissue according to any of the above items, wherein 99% or more of epidermal cells are the first cells, and less than 1% of the epidermal cells are the second cells; and 90% or more of dermal cells are the second cells, and less than 10% of the dermal cells are the first cells.

[30] The skin tissue according to any of the above items, wherein 99% or more of epidermal cells are the first cells, and less than 1% of the epidermal cells are the second cells; and 50% or more of dermal cells are the second cells, and less than 50% of the dermal cells are the first cells.

[31] The skin tissue according to any of the above items, wherein 95% or more of epidermal cells are the first cells, and less than 5% of the epidermal cells are the second cells; and 50% or more of dermal cells are the second cells, and less than 50% of the dermal cells are the first cells.

[41]A grafting material comprising a skin tissue according to any of the above items.

[42] The grafting material according to [41] for use in transplantation in skin grafting to a subject.

[43] The grafting material according to [42], wherein the subject has a skin damage caused by a burn or a wound.

[51] The skin tissue according to any of the above items, wherein the first cells are derived from a human, and the second cells are derived from a nonhuman mammal.

[52] The skin tissue according to [51], wherein the first cells are derived from a human, and the second cells are derived from a pig.

[53] The skin tissue according to any of the above items, wherein the first cells are deficient in any or both of HLA class 1 and class II, whereby tolerance of immune rejection to allogeneic graft is improved as compared with a case without the suppression of expression.

[54] The skin tissue according to [53], wherein the first cells are deficient in both of HLA class 1 and class II, whereby tolerance of immune rejection to allogeneic graft is improved as compared with a case without the suppression of expression.

[55] The skin tissue according to [53], wherein the first cells undergo knockout of any or both of $2 microglobulin and CIITA, whereby tolerance of immune rejection to allogeneic graft is improved as compared with a case without the suppression of expression.

[56] The skin tissue according to [54], wherein the first cells undergo knockout of both of P2 microglobulin and CIITA, whereby tolerance of immune rejection to allogeneic graft is improved as compared with a case without the suppression of expression.

[57] The skin tissue according to any of [53] to [56], wherein one or more members selected from the group consisting of HLA-G, HLA-E, CD47, and PD-Li are forcedly expressed, whereby tolerance of immune rejection to allogeneic graft is improved as compared with a case without the suppression of expression.

[58] The skin tissue according to any one of [53] to [57], wherein one or more or all members selected from the group consisting of HLA-A, HLA-B, and HLA-C are knocked out at only one of the two genes of the locus (rendered HLA pseudo-homozygous), whereby tolerance of immune rejection to allogeneic graft is improved as compared with a case without the suppression of expression.

[61]A product comprising a skin tissue according to any of the above items and a storage solution suitable for storage thereof.

[62] The product according to [61], wherein the storage solution is physiological saline.

[63] The product according to [61], wherein the storage solution is a serum-free medium.

[64] The product according to [63], wherein the storage solution is a scientifically defined medium.

[65] The product according to any of the above items, further comprising any or both of an anti-inflammatory agent and an immunosuppressant.

[65] The product according to any of the above items for use as a graft in skin grafting.

[71]A grafting material comprising a skin tissue according to any of [51] to [54], for use in transplantation to a human subject from which the first cells are derived.

[72]A grafting material comprising a skin tissue according to [53] or [54], the grafting material being for use in transplantation to a human subject allogenic to a human from which the first cells are derived.

[81]A method for producing a skin tissue, a grafting material, or a product according to any of the above items, comprising:
providing a host embryo incapable of developing a mature epidermis;
injecting donor cells having ability to differentiate into a mature epidermis to the host embryo, wherein the injection is performed under conditions suitable for engraftment of the cells to the skin;
allowing the obtained embryo to grow in the uterus of a pseudo-pregnant surrogate mother mammal by grafting to the uterus thereof to obtain an individual having a mature epidermis from the mammal; and
isolating a skin tissue comprising a mature epidermis comprising cells derived from the donor cells, a basement membrane, and a dermis comprising cells derived from the host from the obtained individual.

[91]A method for preparing a nonhuman mammalian individual, comprising
bringing cells having ability to form a chimera and having ability to differentiate into a mature epidermis, or cells committed to an epidermis or cells of epidermal lineage (donor cells) into contact with a body surface of a nonhuman mammalian individual (host) before formation of epidermal basal cells, thereby obtaining a nonhuman mammalian individual having a skin tissue comprising cells of the host and the donor cells.

[92] The method according to [91], wherein the nonhuman mammalian individual is a p63-knockout individual.

[93] The method according to [91] or [92], wherein the cells having ability to form a chimera and having ability to differentiate into a mature epidermis, or the cells committed to the epidermis or cells of epidermal lineage (donor cells) are human cells.

[94] The method according to any of [91] to [93], wherein the nonhuman mammalian individual is a mouse or a pig.

[95] The method according to any of [91] to [94], wherein the cells having ability to form a chimera and having ability to differentiate into a mature epidermis, or the cells committed to the epidermis or cells of epidermal lineage (donor cells) are human cells, and the nonhuman mammalian individual is a mouse or a pig.

[96] The method according to any of [91] to [95], wherein the cells of the host and the donor cells are contained in the dermis.

[97] The method according to any of [91] to [94], wherein the cells of the host and the donor cells are contained in the epidermis and the dermis, respectively.

[98] The method according to any of [91] to [97], wherein the contact is carried out by injecting the donor cells into an amniotic cavity of the host.

[99] The method according to any of [91] to [98], wherein the contact or the injection is performed by transuterine administration.

[101]A method for preparing a nonhuman mammalian individual, comprising bringing cells having ability to form a chimera and having ability to differentiate into a mature epidermis, or cells committed to an epidermis or cells of epidermal lineage (donor cells) into contact with a body surface of a nonhuman mammalian individual (host) before formation of epidermal basal cells, thereby obtaining a nonhuman mammalian individual having an epidermis comprising the donor cells.

[102] The method according to [101], wherein the nonhuman mammalian individual is a p63-knockout individual.

[103] The method according to [101] or [102], wherein the cells or the epidermis having ability to form a chimera and having ability to differentiate into a mature epidermis, or the cells committed to cells of epidermal lineage (donor cells) are human cells.

[104] The method according to any of [101] to [103], wherein the nonhuman mammalian individual is a mouse or a pig.

[105] The method according to any of [101] to [104], wherein the cells having ability to form a chimera and having ability to differentiate into a mature epidermis, or the cells committed to the epidermis or cells of epidermal lineage (donor cells) are human cells, and the nonhuman mammalian individual is a mouse or a pig.

[106] The method according to any of [101] to [105], wherein the contact is carried out by injecting the donor cells into an amniotic cavity of the host.

[107] The method according to any of [101] to [106], wherein the contact or the injection is performed by transuterine administration or administration into an amniotic cavity.

### BRIEF DESCRIPTION OF DRAWINGS

FIG. 1 shows the appearance at E18.5 of a p63-knockout mouse.

FIG. 2 shows a scheme of preparation of a p63-knockout embryo, injection of pluripotent cells (in the drawing, GFP-positive embryonic stem cells (ESCs) are used) to the embryo, transplantation to a pseudo-pregnant surrogate mother mouse, and obtainment of a chimeric embryo.

FIG. 3 shows an optical microscope image of a wild-type (WT) mouse fetus and chimeric embryos of E19.5, and a fluorescence microscope image of observed fluorescence derived from GFP of the embryos. In the fluorescence microscope image, fluorescence derived from GFP mainly from body surfaces was detected.

FIG. 4 shows the left and right appearance of chimeric embryos of E19.5, and fluorescence microscope images of observed fluorescence derived from GFP from body surfaces (left and right) of the embryos. The numbers in the drawings correspond to individual Nos. in Table 1.

FIG. 5 is a diagram of a plot of the relationship between a rate of chimeric contribution (chimerism) of donor cells in the spleen and a body surface area where the epidermis was formed (ratio to the total body surface area).

FIG. 6 shows fluorescent images derived from GFP on a body surface 7 days and 56 days after grafting of the obtained skin tissue. In this experiment, the skin tissue was isolated from a chimeric neonate obtained by the growth of a chimeric embryo, and grafted to a region of another mouse from which a skin tissue was excised.

FIG. 7A shows fluorescence microscope images (GFP, CK8/K18, and p63) of the epidermis of a chimeric embryo.

FIG. 7B is a merged image of the images of FIG. 7A.

FIG. 7C shows a manner in FIG. 7B in which host cells are pushed away by donor cells, and a skin surface was occupied by the donor cells.

FIG. 8A shows fluorescence microscope photographs of a partial body surface of a chimeric embryo of E18.5.

FIG. 8B is a merged image of the images of FIG. 8A. The eliminated host cells were positioned outside the outermost layer of the skin and no longer remained in the skin tissue.

FIG. 9A shows the appearance of chimeric individuals of E16.5 obtained by grafting GFP-positive human HaCaT cells into the amniotic cavity of p6³-knockout mouse embryos of E13.5.

FIG. 9B is an enlarged view of the box of FIG. 9A.

FIG. 9C shows a fluorescence microscope image of the chimeric individuals of FIG. 9A.

FIG. 10A shows the relationship between a chimeric rate of embryos and a surface covered rate of formed skin.

FIG. 10B is a fluorescence microscope image of a skin section of a chimeric embryo prepared from a p63-KO embryo and donor embryonic stem cells.

FIG. 11A shows a fluorescence microscope image of the epidermis of a chimeric embryo prepared from a wild-type embryo and donor embryonic stem cells.

FIG. 11B shows a fluorescence microscope image of the epidermis of a chimeric embryo prepared from a p63-KO embryo and donor embryonic stem cells.

FIG. 12 shows results of engraftment of the skin after grafting of the skin tissue of the present disclosure.

FIG. 13 shows results of construction of a human epidermis on a mouse individual by the method of the present disclosure.

### DESCRIPTION OF EMBODIMENTS

In the present specification, the “subject” refers to a mammal. Examples thereof include, but are not particularly limited to rodents such as mice and rats, livestock animals such as pigs, goats, llamas, sheep, and bovines, pet animals such as dogs and cats, birds such as chickens, and primates such as monkeys. In the present specification, the animal can be a nonhuman animal.

In the present specification, the “somatic chimeric animal” refers to an animal having a tissue or an organ in which cells of a certain individual and cells of another individual (e.g., allogeneic cells, or syngeneic cells or allogeneic cells engineered with one or more genes) coexist at the cell level. In the present invention, the somatic chimeric animal can be obtained by introducing a plurality of (e.g., approximately 10) pluripotent cells to an embryo (e.g., morula or blastocyst). More specifically, various embryos from an embryo at an eight-cell stage to an embryo at a blastocyst stage can be used as the embryo, and a method for introducing pluripotent cells to such an animal embryo is well known to those skilled in the art. In the case of introducing cells to an embryo at a blastocyst stage, the cells can be introduced to, for example, a blastocoel. Early embryos up to a morula stage may be aggregated by the contact of the cells. Alternatively, the somatic chimeric animal may be obtained by administering the cells into an amniotic fluid of a fetus. In this embodiment, cutaneous epithelium cells can be administered into the amniotic fluid. Examples of the pluripotent cells include pluripotent stem cells such as ES cells and iPS cells, and pluripotent cells such as inner cell masses (ICMs). In the present invention, these cells can be introduced to the embryo. The number of pluripotent stem cells to be introduced to the embryo can also be appropriately determined and can be for example, but not particularly limited to, on the order of 3 to 10 for introduction to the embryo. In the present specification, the “somatic chimeric animal” is also simply referred to as a “chimera”.

In the present specification, the “host animal” means an individual, such as an embryo (hereinafter, also referred to as a “host embryo”), to which cells such as pluripotent cells are introduced in preparing a somatic chimeric animal.

In the present specification, the “cells for introduction to an embryo” mean cells (hereinafter, also referred to as “donor cells”) to be introduced to an individual such as an embryo in preparing a somatic chimeric animal.

In the present specification, the “pluripotent cells” mean pluripotent stem cells such as ES cells and iPS cells, and pluripotent cells such as inner cell masses (ICMs). It is known that the pluripotent cells can differentiate into almost all cells of a fetus or an adult.

In the present specification, the “genetic engineering” means genetic modification. In the present specification, the modification of gene expression means modification that causes enhancement or attenuation of gene expression. The attenuation of gene expression may be performed by gene disruption.

In the present specification, the “genetic modification” means that a gene is different from a wild type, and includes artificial modification. Typical examples of the genetic modification include transgenesis and knockout. The knockout is not particularly limited, and, for example, loss of function in a target gene is induced by introducing a mutation such as nonsense mutation or frameshift mutation to the target gene or by disrupting a control region of the target gene. In the knockout, a foreign gene such as a marker gene may be introduced to the inside of the target gene or so as to replace the target gene.

In the present specification, the term “comprise” means that a described component is included while a component not described may or may not be included. In the present specification, the term “consist of” means that a described component is included while an unavoidable inclusion of a component not described is acceptable.

In the present specification, the “epithelium” is a tissue that covers the outer surface and a cavity of the body or a lumen of an organ, etc., in an animal individual. In the present specification, the “skin” is a tissue that covers the outer surface of an animal individual. The skin is constituted by the epithelial epidermis and the dermis of connective tissue located beneath the epidermis.

The epidermis is composed mainly of keratinocytes. In the epidermis, keratinocytes in different forms (e.g., a basal layer (layer consisting of basal cells), a spinous cell layer, a granular cell layer, and a stratum corneum) depending on the state of maturation are arranged in layers because keratinocytes resulting from a layer of basal cells including stem cells of keratinocytes migrate to the surface side as maturated. The keratinocytes account for approximately 95% of the epidermis, and the remaining 5% includes Langerhans cells and Merkel cells, etc. and participates in immunity and perception. The epidermis is usually absent in blood vessels and nerves.

The skin has the basement membrane immediately beneath the epidermis. The basement membrane has the basal lamina and the lamina lucida and lies between the epidermis and the dermis. Basal cells are attached to the basal lamina via the lamina lucida on a surface opposite to the dermis (on the epidermis side), thereby actively causing cell division. The epidermis and the dermis are strongly joined via the basement membrane. It is considered that keratinocytes are strongly connected through a structure responsible for cell-cell adhesion, such as desmosome; and the basement membrane are joined to basal cells via the lamina lucida through hemidesmosome.

The dermis is a cutaneous layer having a dermal papillary layer and a dermal reticular layer between the epidermis and subcutaneous tissue. The dermis is composed mainly of fibrous connective tissue. Approximately 70% of the dermis is occupied by collagen, and the remaining part is constituted by fibers such as elastic fiber (elastin), extracellular matrix, and hyaluronic acid. The dermis has organs such as blood vessels, lymph nodes, sweat glands, and follicles. The dermis also has fibroblasts that form collagen fiber, macrophages involved in immunity or inflammation, mast cells, and plasma cells. Subcutaneous tissue is further located behind the dermis.

The epidermis is derived from the ectoderm, while the dermis is occupied mainly by fibroblasts. The fibroblasts are derived from the mesoderm. Thus, the epidermis and the dermis differ embryologically. The dermis has blood vessels and nerves in addition to the fibroblasts. It is considered that the blood vessels are derived from the endoderm; and the nerves are derived from the ectoderm.

The human epithelium and skin are considered to generally have the structures described above.

The present disclosure is capable of providing a skin tissue. The skin tissue comprises an epidermis, a basement membrane, and a dermis. The epidermis is attached to one surface of the basement membrane (basal cells on this surface), and the dermis is attached to the other surface (basal lamina) of the basement membrane. lamina lucida may lie between the dermis and the basal lamina. The epidermis, the basement membrane, and the dermis are layered to form a structure, whereby the skin tissue is capable of having higher mechanical strength than that of a structure of the epidermis alone. The skin tissue provided by the present disclosure may be used as a grafting material for skin grafting. In the grafting material, the epidermis is preferably autologous or allogeneic, while the dermis may comprise allogeneic or xenogeneic cells. In a preferred embodiment, the epidermis is autologous. More preferably, the autologous cells are free from foreign substances (e.g., foreign genes, products of foreign genes, and other substances having antigenicity). This is because such a grafting material can be engrafted to an individual after grafting.

In the skin tissue of the present disclosure, the epidermis comprises first cells, and the dermis comprises second cells. The epidermis is derived from the ectoderm, and the dermis is derived from the mesoderm. Thus, the epidermis and the dermis differ embryologically. Thus, the epidermis comprises ectodermal cells, and the dermis comprises mesodermal cells. Thus, the first cells may be ectodermal cells, and the second cells may be mesodermal cells. The first cells have ability to differentiate into mature epidermal cells, while the second cells have no ability to differentiate into mature epidermal cells by, for example, genetic modification or gene knockdown. In a preferred embodiment, the second cells have no ability to differentiate into any or all cells selected from the group consisting of basal cells, cornified cells, granular cells, and spinous cells. In an embodiment, the second cells may have ability to differentiate into basal cells or may have no ability to differentiate into basal cells. According to Examples mentioned later, when the second cells have no ability to differentiate into mature epidermal cells, the cells are capable of being replaced with the first cells even if having ability to differentiate into basal cells. Thus, the basal cells may include the first cells. In a preferred embodiment, the first cells have an allogeneic relationship with the second cells. In a preferred embodiment, the first cells have a xenogeneic relationship with the second cells. For example, the first cells may be derived from a human, and the second cells may be derived from a mammal selected from the group consisting of a primate, a pig, a goat, sheep, a llama, and a bovine (e.g., the second cells may be from a pig). In the present specification, the “cells having ability to differentiate into mature epidermal cells” are used in a meaning including undifferentiated cells having the ability to differentiate into mature epidermal cells as well as cells that have differentiated into mature epidermal cells.

In a preferred embodiment, the first cells may have ability to differentiate into one or more or all cells selected from the group consisting of basal cells, cornified cells, granular cells, and spinous cells. In a preferred embodiment, the second cells have low ability to differentiate into mature epidermal cells, or preferably have no such ability. In a preferred embodiment, the first cells have ability to differentiate into one or more or all cells selected from the group consisting of basal cells, cornified cells, granular cells, and spinous cells, and the second cells have low ability to differentiate into any or all cells selected from the group consisting of cornified cells, granular cells, and spinous cells, or preferably have no such ability. In this embodiment, the second cells may have ability to differentiate into basal cells.

In a preferred embodiment, the first cells may have ability to differentiate into one or more or all cells selected from the group consisting of basal cells, cornified cells, granular cells, and spinous cells, and the second cells may be cells having loss of function in p63. In a preferred embodiment, the first cells have p63 gene having a normal function. This enables substantially all cells of the epidermis to be derived from the first cells. The dermis is capable of becoming a chimera of the first cells and the second cells.

The epithelium of a p63-deficient individual has a single layer of cells expressing keratin K8/K18 on the basement membrane so that further development does not progress (see Shalom-Feuerstein et al., Cell Death Differ., 18: 887-896, 2011, which is incorporated herein by reference in its entirety). When donor cells having ability to differentiate into an epidermis is introduced to a p63-deficient embryo which is then developed, host cells on the basement membrane are almost completely removed and almost 100% or 100% of the epidermis becomes cells derived from the donor cells (the epidermis consists of cells derived from the donor cells). The skin thus obtained is capable of being preferably used as the skin of the present disclosure. Recent advancement in genome editing technology allows for genome editing of embryos of various mammals including primates, livestock, and pet animals and also allows for disruption of p63. Thus, the skin of the present disclosure in which the first cells are human cells and the second cells are from any mammal such as a primate, livestock, or a pet animal can be obtained by appropriately preparing a p63-KO animal embryo or individual having an epidermis consisting of human cells.

The loss of function in p63 may be generated by disruption or knockdown of p63 gene. The disruption of the p63 gene can be based on one or more members selected from, for example, disruption of a control region of the p63 gene, introduction of nonsense mutation to the p63 gene, introduction of frameshift mutation thereto, introduction of missense mutation thereto, and introduction of knockout cassette to a translated region. The disruption of the p63 gene or the introduction of a knockout cassette can be appropriately achieved by those skilled in the art using, for example, genome editing technology. Examples of the genome editing technology include TALE nuclease (TALEN), zinc finger nuclease (ZFN), and CRISPR/Cas9 system. These techniques can be used in the formation of cells having the loss of function in p63. More specifically, a target region is established in the p63 gene, and TALEN, ZFN, or CRISPR/Cas9 designed so as to cleave the target region is introduced to cells to cleave the target region. The cleaved ends are rejoined when the cleaved target region is repaired by a self-repair mechanism of the cells. However, in this course of repair, nonsense mutation or frameshift mutation can occur because nucleotides can be randomly lost from the nucleotide sequence. Also, in the presence of donor DNA comprising a knockout cassette in this course of repair, genomic DNA integrates the donor DNA, thereby attaining disruption of the gene. The donor DNA has an upstream homology arm having a sequence homologous to an upstream region of the cleavage site, and a downstream homology arm having a sequence homologous to a downstream region of the cleavage site, and comprises a knockout cassette between the upstream homology arm and the downstream homology arm. The knockout cassette may comprise, for example, a selective marker gene (e.g., a drug selection marker gene or a gene encoding a visible marker such as a fluorescent protein) for selecting cells in which the knockout cassette is inserted. In this way, those skilled in the art can obtain cells having a disrupted gene using genome editing technology designed such that a target region can be appropriately cleaved. Alternatively, the introduction of the knockout cassette may be performed by traditional homologous recombination. Whether or not the p63 gene has been disrupted can be confirmed by the sequencing of the target region. Whether or not the p63 gene has been disrupted can also be confirmed by testing whether or not the cells have ability to differentiate into a mature epidermis. Whether or not the cells have ability to differentiate into a mature epidermis can be confirmed by obtaining a p63-knockout individual using ES cells having the disrupted gene (p63-knockout ES cells) or fertilized eggs having the disrupted gene, and confirming the formation of an epidermis thereof.

In an embodiment, the present disclosure provides an isolated skin tissue comprising an epidermis, a basement membrane, and a dermis, wherein the epidermis is attached to one surface of the basement membrane, and the dermis is attached to the other surface of the basement membrane; and the epidermis comprises first cells, and the dermis comprises second cells, wherein the second cells have no ability to differentiate into mature epidermal cells. In this context, in a preferred embodiment, the first cells may have p63 gene having a normal function, and the second cells may have loss of function in p63, for example, may have disrupted p63 gene.

In this embodiment, 95% or more, 96% or more, 97% or more, 98% or more, or 99% or more of cells of the epidermis (which are particularly keratinocytes and may comprise a basal layer (layer consisting of basal cells), a spinous cell layer, a granular cell layer, and a stratum corneum) may be the first cells. Also, 60% or more, preferably 70% or more, more preferably 80% or more, further preferably 90% or more, still further preferably 95% or more, 96% or more, 97% or more, or 98% or more, especially preferably 99% or more, of the dermis may consist of the second cells. The rate of contribution of the first cells in the dermis presumably depends on the ability of the first cells to form a chimera in an embryo. Even low ability of the first cells to form a chimera in an embryo is preferred because the rate of chimeric contribution of the first cells in keratinocytes can maintain a high value whereas the rate of chimeric contribution in tissues other than the epidermis can be kept low.

In a preferred embodiment, in the skin tissue of the present disclosure, 95% or more of cells of the epidermis are the first cells, and 75% or more of cells of the dermis are the second cells. In a preferred embodiment, in the skin tissue of the present disclosure, 99% or more of cells of the epidermis are the first cells, and 95% or more of cells of the dermis are the second cells. In a preferred embodiment, in the skin tissue of the present disclosure, 99% or more of cells of the epidermis are the first cells, and 99% or more of cells of the dermis are the second cells.

In the skin tissue of the present disclosure, the first cells are derived from donor cells described below, and the second cells are derived from a host embryo described below. Thus, technical requirements for the donor cells and the host embryo are applicable to technical requirements for the first cells and the second cells, respectively.

The present disclosure provides a composition for use in preparing the skin tissue of the present disclosure in which 99% or more of cells of the epidermis are the first cells and 99% or more of cells of the dermis are the second cells, the composition comprising the first cells. The present disclosure provides a composition for use in preparing the skin tissue of the present disclosure in which 99% or more of cells of the epidermis are the first cells and 99% or more of cells of the dermis are the second cells, the composition comprising the second cells. The present disclosure provides a kit for use in preparing the skin tissue of the present disclosure in which 99% or more of cells of the epidermis are the first cells and 99% or more of cells of the dermis are the second cells, the kit comprising the first cells and the second cells. In the kit, the first cells and the second cells are preferably contained in separate containers, respectively.

The present disclosure is capable of providing a grafting material comprising the skin tissue of the present disclosure. The grafting material of the present disclosure may be used for repairing a wound. The grafting material of the present disclosure may be used for treating a body surface having a burn. If necessary, damaged skin is excised, and the grafting material of the present disclosure may be grafted to the body surface from which the skin has been excised. The grafting material of the present disclosure can also be used in the treatment of skin diseases such as refractory ulcer and bullosis. The grafting material of the present disclosure can be further used in the screening of a pharmaceutically active ingredient (e.g., a compound) for use in the treatment of a burn, beauty care, skin tumor, or the like.

The present disclosure provides a method for preparing the skin tissue of the present disclosure (hereinafter, referred to as a “method for producing the skin tissue of the present disclosure”).

The method for producing the skin tissue of the present disclosure comprises providing a host embryo. The host embryo is an embryo having low ability to differentiate into mature epidermal cells, or preferably having no such ability. In an embodiment, the host embryo has undergone genetic engineering (e.g., gene knockout or knockdown) so as to have low ability to differentiate into mature epidermal cells, or preferably have no such ability. The host embryo is a supply source of the second cells in the skin tissue of the present disclosure. The method for producing the skin tissue of the present disclosure may comprise injecting donor cells having ability to differentiate into mature epidermal cells to the host embryo to form a chimeric individual. The injection may be performed under conditions suitable for engraftment of the donor cells to the skin.

The donor cells may be, for example, pluripotent cells. The donor cells may also be, for example, cells (e.g., stem cells and progenitor cells) having ability to differentiate into a mature epidermis, or cells committed to epidermis or cells of epidermal lineage (e.g., epidermal stem cells and keratinocytes such as basal cells, spinous cells, granular cells, and cornified cells). The donor cells may be preferably pluripotent cells. The donor cells may also be preferably cells having ability to differentiate into a mature epidermis, or cells committed to epidermis or cells of epidermal lineage, particularly, epidermal stem cells or basal cells. All the cells having ability to differentiate into a mature epidermis can be used as the donor cells except for the case where no epidermis derived from the donor cells is formed by marked inhibition of engraftment. Cells having various stages of differentiation may be used as the donor cells as long as the cells have ability to differentiate into a mature epidermis, as is evident from the fact that the skin having an epidermis consisting of the donor cells can be obtained by using pluripotent stem cells as the donor cells; and the skin having an epidermis consisting of the donor cells can also be obtained by using HaCaT cells as the donor cells. The donor cells can then be brought into contact with the host embryo before formation of basal cells (e.g., CK8/K18-positive cells) of the host embryo, whereby basal cells and an epidermis can be formed from cells derived from the donor cells. The donor cells may be autologous cells or allogeneic cells and are preferably autologous cells, more preferably unmodified autologous cells. The term “unmodified” means that the cells have not undergone transformation (e.g., modification by gene recombination). When the donor cells are allogeneic cells, the donor cells may preferably be tolerant to immune rejection (i.e., tolerance of immune rejection is improved). The donor cells are preferably deficient in any, or preferably both, of HLA class I and class II in genomic DNA thereof, whereby tolerance of immune rejection in allogeneic graft is improved. The deficiency in HLA class I may be performed by knockout of β2 microglobulin or knockout of HLA class I. The deficiency in HLA class II may be performed by knockout of CIITA or knockout of HLA class II. One or more members selected from the group consisting of HLA-G, HLA-E, CD47, and PD-Li are overexpressed or forcedly expressed, whereby tolerance of immune rejection to allogeneic graft can be improved as compared with a case without the suppression of expression. One or more or all members selected from the group consisting of HLA-A, HLA-B, and HLA-C are knocked out at only one of the two genes of the locus (rendered HLA pseudo-homozygous), whereby tolerance of immune rejection to allogeneic graft can be improved as compared with an HLA heterozygote. A suicide gene linked under the control of an inducible promoter may be knocked in the donor cells such that it is possible to actively remove the donor cells after grafting for some reason.

In a preferred embodiment, a plurality of donor cells may be brought into contact with (or administered to) one host embryo. The number of donor cells to be administered to one host embryo may be, for example, on the order of 3 to 10, for example, 3 to 5, 5 to 7, or 8 to 10.

The donor cells may be cells having low ability to contribute to a chimera (ability to form a chimera). The donor cells may have ability to contribute to a chimera with a rate of chimeric contribution of, for example, 50% or less, 40% or less, 30% or less, 20% or less, 15% or less, 10% or less, 5% or less, 4% or less, 3% or less, 2% or less, or 1% or less as long as the donor cells have ability to form a chimera. The ability to contribute to a chimera may be determined as the ratio of cells derived from the donor cells to all cells in an individual or a portion (e.g., a particular organ) of the individual, by injecting the donor cells to a normal embryo (e.g., a normal embryo at a blastocyst stage) to develop a chimeric individual. When the donor cells have low ability to contribute to a chimera, the donor cells are capable of making only limited contribution to tissues other than the epidermis. This is preferred because ethical problems can be reduced.

When the host embryo is at an eight-cell stage to a blastocyst stage, the donor cells can be mixed with cells of the host embryo or injected to a blastocoel of the host embryo. When the host embryo is at a blastocyst stage or a later stage, the donor cells can be administered into an amniotic cavity, whereby the donor cells can be brought into contact with a surface of the host embryo. These injection methods are illustrative, and various other injection methods are present. Those skilled in the art can appropriately select or determine an injection method and use the injection method in the method of the present disclosure.

The host embryo to which the donor cells have been injected or administered (in the present specification, referred to as a “chimeric embryo”) can be transferred to the uterus of a pseudo-pregnant surrogate mother mammal, engrafted, and then allowed to grow. The pseudo-pregnant surrogate mother mammal is preferably the same animal species as that of the host embryo. This can promote engraftment of the host embryo to the uterus.

The obtained chimeric embryo may have a skin tissue comprising an epidermis, a basement membrane, and a dermis. The obtained chimeric embryo is allowed to further grow, whereby the epidermis becomes mature and develops into an epidermis having a stratum corneum, a granular cell layer, and a spinous cell layer. A skin tissue comprising an epidermis, a basement membrane, and a dermis, preferably a skin tissue comprising a mature epidermis, a basement membrane, and a dermis, can be isolated from the obtained chimeric embryo or a chimeric individual obtained by further growth of the chimeric embryo. The isolation can be performed by a physical excision technique.

The isolated skin tissue may be stored in an environment (e.g., under an oxygen concentration, a carbon dioxide concentration, and a temperature condition) suitable for storage, for example, in an appropriate solution such as physiological saline or a liquid medium. The oxygen concentration may be, for example, approximately 20%, the carbon dioxide concentration may be 0 to approximately 5%, and the temperature condition may be 0° C. to approximately 37° C. (particularly, a body temperature).

In an embodiment, the host embryo may be a nonhuman mammalian embryo. In an embodiment, the donor cells may be human cells or nonhuman mammal cells. In an embodiment, the host embryo may be a pig embryo, and the donor cells may be human cells. As shown in Examples mentioned later, cell engraftment has been confirmed between mice and humans having a longer evolutionary distance than that between pigs and humans. Thus, the human cells can be introduced to embryos of many nonhuman mammal species to form chimeric embryos or chimeric individuals. The human cells may be from the skin of a recipient or may be preferably obtained from an organoid having a human epidermis. The human cells can be obtained from, for example, but not particularly limited to, an organoid obtained by a method described in Lee, J. et al., Nature 582, 399-404, 2020. In this way, human cells (particularly, cells having ability to differentiate into a mature epidermis) autologous to a recipient can be obtained. Allogeneic cells or xenogeneic cells, when introduced to the host embryo before establishment of an immune system, are capable of being engrafted to the embryo without receiving immune rejection. However, inflammation may occur in the skin or the like after birth (see WO2018/139502A). Hence, the skin tissue of the present disclosure can be treated with an anti-inflammatory agent or an immunosuppressant before inflammation is found in the skin tissue after birth. The skin tissue of the present disclosure may be treated with an anti-inflammatory agent or an immunosuppressant after inflammation is found in the skin tissue after birth.

Examples of the immunosuppressant include, but are not particularly limited to, cyclosporin and tacrolimus as calcineurin inhibitors, rapamycin and everolimus as mTOR inhibitors, azathioprine, mizoribine, methotrexate, mycophenolate mofetil, and leflunomide as antimetabolites, and cyclophosphamide as an alkylating agent, any of which can be used in the present invention. Examples of the anti-inflammatory agent include, but are not particularly limited to, steroidal anti-inflammatory drugs (SAIDs) and nonsteroidal anti-inflammatory drugs (NSAIDs), any of which can be used in the present invention. Examples of the steroid include cortisol, prednisolone, triamcinolone, beclomethasone, betamethasone, fluticasone, dexamethasone, and hydrocortisone, any of which can be used in the present invention. Other examples of the anti-inflammatory agent include inflammatory cytokine inhibitors such as anti-inflammatory cytokine antibodies, for example, anti-TNF-α antibodies, and antibodies against soluble cytokines, for example, soluble TNF receptors, any of which can be used in the present invention.

In an embodiment of the present invention, a steroidal anti-inflammatory agent having a function as the immunosuppressant and a function as the anti-inflammatory agent may be preferably used. In the present invention, it can be expected that sufficient effects can be obtained by merely suppressing immune response or inflammation.

The skin tissue of the present disclosure may be stored in physiological saline or a medium containing the anti-inflammatory agent and/or the immunosuppressant and grafted to an individual in need of the skin tissue (in the present specification, referred to as a “recipient”) (e.g., a human). Owing to the presence of the dermis, the skin tissue thus grafted is capable of regenerating the skin on a body surface of the recipient in the presence of the anti-inflammatory agent and/or the immunosuppressant. It can be expected that on the body surface of the recipient, cells derived from the host embryo are removed and replaced with cells of the recipient, as the time passes. Thus, it can be expected that the skin is regenerated in the recipient after a lapse of a sufficient time after grafting.

Thus, the donor cells may be preferably cells derived from a recipient (preferably induced pluripotent cells derived from a recipient from the viewpoint of easy availability), and a grafting material comprising the obtained skin tissue may be used as a graft in the recipient.

### Examples

### Material and Experimental Method

### 1) Pluripotent Stem Cell

The mouse embryonic stem cells (ESCs) used were mouse ESCs (line name: B6ES2 and SGE2) established from C57BL/6N and C57BL/6N-Tg (CAG-EGFP) mice, respectively. The rat ESCs used were rat ESCs (line name: BN2i-4) established from BN rat. The ESCs were cultured using mouse embryonic fibroblasts treated with mitomycin C as feeders. The medium used for culture was N2B27 medium supplemented with 1 μM PD0325901, 3 μM CHIR99021, and 1000 U/ml mouse leukemia inhibitory factor (LIF).

### 2) HaCaT Cell

The HaCaT cells were purchased from Cell Lines Service GmbH and cultured in Cnt-07 (CELLnTEC Advanced Cell Systems AG) medium. For fluorescent labeling, an EGFP expression cassette under the control of CAG promoter was introduced to the BN2i-4 and HaCaT cells using lentivirus vectors. Only GFP(+) populations after introduction were separated in a flow cytometer (BD, FACS Aria III), and the resulting GFP-positive cells were used in experiments. In the description below, the fluorescently labeled cells are referred to as “BN2i-4-EGFP” and “HaCaT-EGFP”.

### 3) Animal

Slc:ICR mice, C57BL/6N-Tg (CAG-EGFP) mice, DBA/2CrSlc mice, and BN/SsNSlc rats were purchased from Japan SLC, Inc. and used in experiments. All the animal experiments were carried out in conformity with the prescribed guideline after obtainment of approval of the ethical committee of The Institute of Medical Science, The University of Tokyo.

### 4) Preparation of p63 Mutation Chimeric Animal

In order to obtain p63-knockout embryos, fertilized eggs having small deletion/insertion mutation were prepared by fertilized egg CRISPR/Cas9 genome editing. First, one-cell stage fertilized eggs were collected from ICR females (0.5 dpc) confirmed to have the vaginal plug after natural mating. The fertilized eggs were cultured in KSOM/AA medium for several hours and electroporated in a ribonucleoprotein (RNP)-containing electrode solution. The RNP-containing electrode solution was prepared by mixing Opti-MEM medium (Thermo Fischer Scientific Inc.) with 2.94 μM (final concentration) sgRNA (IDT, Inc., Alt-R™ CRISPR-Cas9 sgRNA, which recognizes the nucleotide sequence CACGGATAACAGCGCCCTGT (SEQ ID NO:1) in p63 exon 5) and 0.61 μM (final concentration) Cas9 protein (IDT, Inc., Alt-R™ S.p. Cas9 Nuclease). In order to obtain p63 mutation chimeras, the lamina lucida of fertilized eggs cultured up to an 8 cell stage after disruption of p63 gene was punctured (Piezo or laser puncturing apparatus), and the donor ESCs (SGE2 or BN2i-4-CAG-EGFP) were microinjected at 2 to 5 cells per embryo to the perivitelline space. Chimeric embryos after injection were cultured to a blastocyst stage in KSOM/AA medium and embryonically transplanted to the uterus of pseudo-pregnant mice to obtain chimeric fetuses.

For skin grafting experiments, fertilized eggs (BDF1-EGFP) obtained by in vitro fertilization of C57BL/6N-Tg (CAG-EGFP) eggs and DBA2 sperm were used in the experiments instead of the ICR mouse fertilized eggs. C57BL/6N-derived B6ES2 was injected to these fertilized eggs to obtain chimeric fetuses.

### 5) Genotyping

A portion of the tails of the p63 mutation mice was collected, permeated with a proteinase K-containing lysis buffer (20 mM Tris-HCl, 100 mM NaCl, 5 mM EDTA, 0.1% SDS), and lysed by heating at 60° C. for 5 minutes to 24 hours, followed by the inactivation of proteinase K by heating at 98° C. for 2 minutes to obtain genomic DNA extracts. Primers (CACGTTTGTACAAGCCAGAACTTA (SEQ ID NO:2) and TCTTTTGGTCTTCCCGAGCCT (SEQ ID NO:3)) were designed so as to flank the mutation region, and the target sequence was amplified through PCR reaction, and then, was confirmed by Sanger sequencing and genotyped by TIDE analysis (doi:10.1093/nar/gku936). For chimeric fetuses, host spleen lymphocytes (GFP−/CD45+/DAPI− or GFP+/CD45+/DAPI−) were separated in a flow cytometer and subjected to similar genotyping. In subsequent experiments, individuals in which frameshift mutation alleles or extensive deletion alleles of 50 or more base pairs accounted for 90% or more of entire alleles were used as subjects to be analyzed.

### 6) In Utero Injection

The uterus was exposed by opening the abdomen of the p63 mutation embryo-grafted pseudo-pregnant mice (E13.5) under isoflurane inhalation anesthesia. The uterine wall was punctured with a glass needle, and HaCaT-EGFP was injected, together with 5 μl of a medium, into the amniotic cavity, followed by the closing of the abdomen. On 3 days after grafting, p63 mutation fetuses were harvested. The engraftment of the cells was observed under a fluorescence microscope, and skin pieces were collected and pathologically evaluated.

### 7) Skin Grafting

In order to collect skin grafts, first, fetuses were harvested by Caesarean section of the p63 mutation chimeric embryo-grafted pseudo-pregnant mice (E18.5). This corresponds to the previous day of birth because mice usually give birth on 19.5 pregnancy days. The chimeric fetuses were confirmed under a fluorescence microscope, and skin pieces were collected from the p63 mutation chimeric fetuses and manually prepared into split-thickness grafts with scissors. Full-thickness lateroabdominal skin was removed from recipient mice (8- to 12-week-old C57BL/6N mice) or recipient rats (3- to 6-week-old BN rats), and the grafts were sutured on them with nylon thread. Gauze was placed on the skin grafts and tied over with nylon thread. The whole circumferences of the trunks of the mice after tie-over were fixed with tape. On 7 days after grafting, the fixation was removed, and the grafted portions were then observed over time to evaluate engraftment. Control groups of homografting experiments were assigned to a group in which the skin of a p63 mutation chimeric fetus was grafted to BDF1-GFP (positive control 1), a group in which the skin of a WT-chimeric fetus prepared by injecting B6ES2 to a BDF1-EGFP fertilized egg was grafted to C57BL/6N-Tg (CAG-EGFP) (negative control 1), a group in which the skin of a BDF1-EGFP fetus was grafted to BDF1-EGFP (positive control 2), and a group in which the skin of a BDF1-EGFP fetus was grafted to C57BL/6N-Tg (CAG-EGFP) (negative control 2), and subjected to skin grafting experiments. In xenogeneic skin grafting experiments, tacrolimus (calcineurin inhibitor; immunosuppressant mainly for T cell immunity) was intraperitoneally administered at 0.5 g per g of rat body weight from the skin grafting day to 14 days after surgery. Control groups were assigned to a group in which C57BL/6N-Tg (CAG-EGFP) was grafted to BN rats (negative control), and a group in which the skin of a BN fetus was grafted to BN rats (positive control), and subjected to skin grafting experiments.

### 8) Chimerism Analysis

Chimerism analysis of the epidermis (keratinocytes and melanocytes), the dermis (fibroblasts), and spleen lymphocytes was conducted by flow cytometry. The epidermis was collected by leaving the skin standing in CnT-07 medium supplemented with 25 U/ml (final concentration) Dispase II (Thermo Fischer Scientific Inc.) at 4° C. for 12 hours, and removing the dermis from the resulting skin with the basement membrane thus selectively treated. In a pan-hematopoietic cell marker CD45-negative and dead cell staining PI-negative epidermal cell fraction, CD49f(+) cells were regarded as keratinocytes, and CD117(+) cells were regarded as melanocytes. Chimerism was measured from a GFP positive ratio. The keratinocytes thus obtained presumably include basal cells, granular cells, spinous cells, and cornified cells. As for the dermis, chopped dermal pieces were cultured in a dish supplemented with DMEM high glucose+10% FBS for several days, and attached cells were analyzed. The spleen was pipetted and then hemolyzed with an ACK buffer, and CD45(+) cells were analyzed as leukocytes.

### 9) Histological Analysis

Chimeric fetuses (E8.5, E14.5, and E18.5) and the skin after skin grafting (14, 28, and 90 days after surgery) were collected and dipped overnight in 4% PFA. Paraffin blocks and sections were prepared by routine procedures. For immunostaining, deparaffinization was performed at stages with xylene and ethanol, and antigen retrieval was performed with a citrate buffer (121° C., 20 min). Each primary antibody was added into a 0.2% triton solution, reacted overnight, and washed. Then, a fluorescently labeled secondary antibody was reacted therewith at room temperature for 1 hour for staining. The prepared specimens were photographed under a fluorescence microscope (Keyence BZ-9000) or a confocal laser scanning fluorescence microscope (Fujifilm FV3000).

### Results

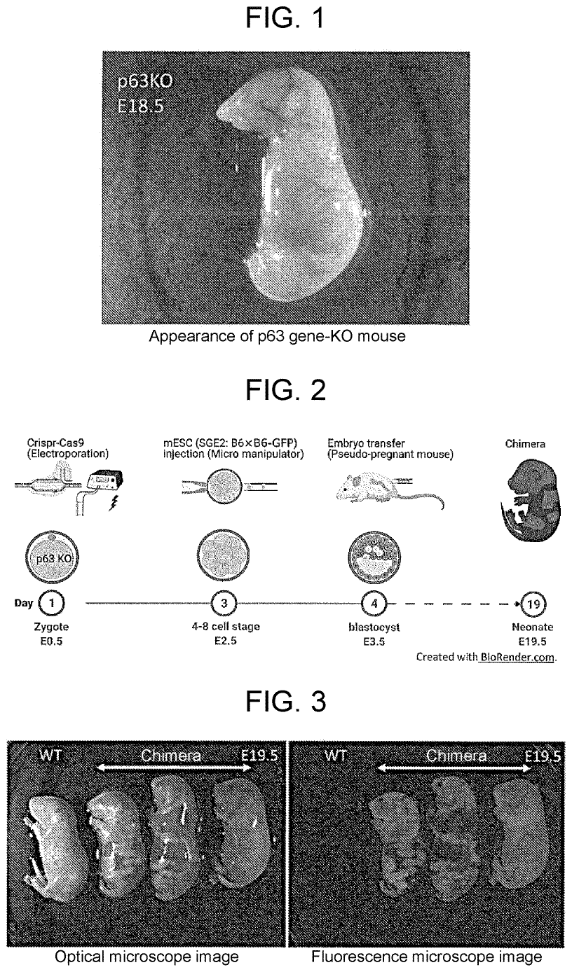

p63-knockout mouse models manifest blood vessels of the epidermis having a partial or complete lack of the squamous epithelium (see Mills et al., Nature, 398: 708-713, 1999; and Yang et al., Nature, 398: 714-718, 1999, which are incorporated herein by reference in their entirety). p63-deficient epithelium remains in a state having a single layer of non-proliferative cells expressing K8/K18, and the development no longer progresses (Shalom-Feuerstein et al., Cell Death Differ., 18: 887-896, 2011, which is incorporated herein by reference in its entirety). In the present Examples, a method was used which involved causing deficiency in p63 gene by CRISPR/Cas9 targeting p63 exon 5, thereby obtaining p63-knockout embryos (see FIG. 1).

An experiment was conducted to obtain chimeric individuals by introducing normal pluripotent stem cells to the p63-knockout mouse embryos that were not completely deficient in the epidermis. Specifically, as shown in FIG. 2, CRISPR/Cas9 system targeting p63 was introduced to a p63-knockout embryo of E0.5. On E2.5, GFP-positive mouse ESCs were injected as pluripotent stem cells to the blastocoel. Then, the cells were allowed to grow to a blastocyst stage of E3.5 and grafted to the uterus of a pseudo-pregnant surrogate mother mouse. On E19.5, the obtained individual (chimeric individual) was observed under an optical microscope and under a fluorescence microscope.

The results were as shown in FIG. 3. A wild type and three chimeric individuals were arranged. The chimeric individuals were confirmed to include individuals having a portion where the epidermis was formed and a portion where the epidermis was not formed (the second and third individuals from the left), and an individual in which the epidermis was almost completely formed (the first individual from the right).

The relationship between the ability (%) of pluripotent stem cells to form a chimera and the area (%) of a regenerated epidermis was examined. The ability of pluripotent stem cells to form a chimera was determined as the percentage of GFP-positive donor cells based on all cells in spleen lymphocytes obtained from each chimeric individual. The ability of pluripotent stem cells to form a chimera was also determined as the percentage of GFP-positive donor cells based on all cells of keratinocytes obtained from each chimeric individual. The area of a regenerated epidermis was determined as the percentage of the area of a GFP-positive epidermis based on the total surface area of an individual. The results were as shown in Table 1.

TABLE 1
Rate of contribution of described donor cell to obtained chimeric embryo of E19.5

| Individual No. | 1 | 2 | 3 | 4 | 5 | 6 | 7 | 8 | 9 |
|---|---|---|---|---|---|---|---|---|---|
| Lymphocyte | 1.5 | 0.9 | 0.0 | 20.1 | 21.4 | 12.6 | 1.0 | 0.1 | 41.7 |
| Keratinocyte | 99.9 | 99.6 | 0.0 | 99.7 | 99.8 | 98.5 | 99.8 | 99.4 | 99.8 |
| GFP-positive area | 25.9 | 54.9 | 0.0 | 88.1 | 63.4 | 100.0 | 29.9 | 35.0 | 100.0 |
| of epidermis | | | | | | | | | |
| (%) | | | | | | | | | |

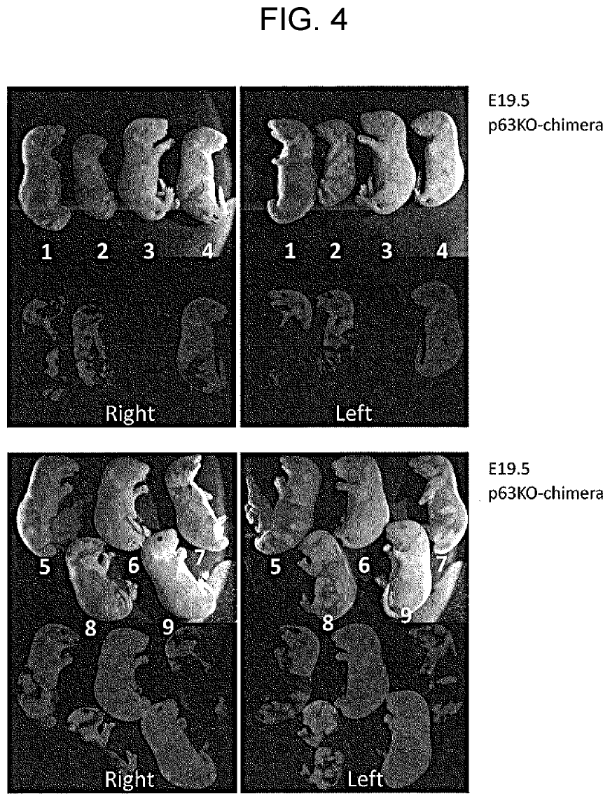

Remarks: in Table 1, the individual Nos correspond to Nos assigned to the individuals of FIG. 4.

As shown in Table 1, the ratio of cells derived from the donor cells in spleen lymphocytes was a very low value, whereas the ratio of the area of a GFP-positive epidermis to the epidermis was substantially large. This revealed that donor cells made large contribution to the epidermis even when donor cells having low ability to form a chimera were used. In all the individuals, a mature epidermis was absent on a surface where no GFP-positive epidermis was formed (see, for example, FIG. 4).

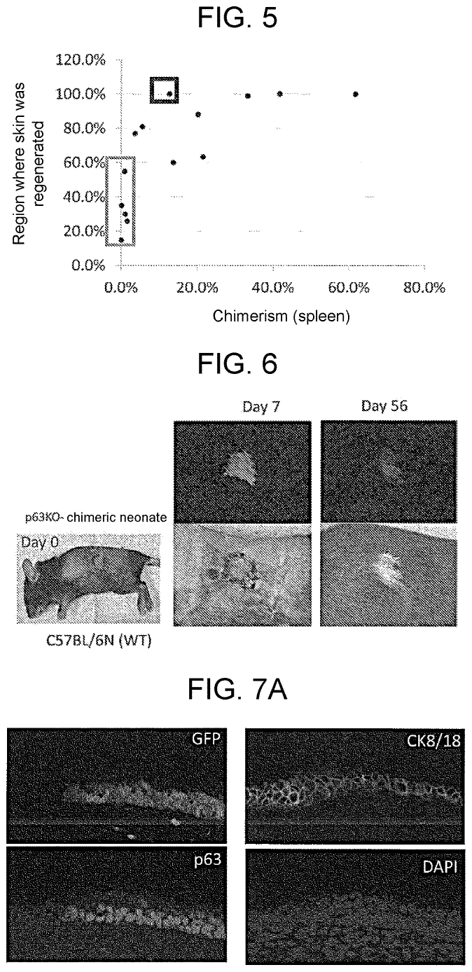

FIG. 5 shows a graph in which the results of Table 1 were plotted. As shown in FIG. 5, even donor cells having low ability to form a chimera, which rarely form a chimera in an organ (here, the spleen) other than the skin, evidently made strong contribution to the skin.

The ratio of GFP-positive donor cells to keratinocytes in the skin was almost 100%, revealing that the formed epidermis was basically derived from the donor cells. This suggested that donor cells form the epidermis by eliminating immature epidermal cells formed in a host.

The skin of the chimeric individuals was histologically analyzed. As a result, the epidermis and the follicles were found to be derived from only the donor cells. By contrast, the dermis was found to be in a chimeric state of the donor cells and the host cells (derived from a p63-KO embryo).

GFP-positive skin (comprising the epidermis and the dermis) of the obtained chimeric individuals was excised and grafted to the skin of wild-type C57BL/6N mice. The grafted skin portions were observed 7 days after grafting. As a result, as shown in FIG. 6, the presence of GFP-positive cells in the grafted portions was found. These GFP-positive cells were also able to be observed 56 days after grafting (see FIG. 6).

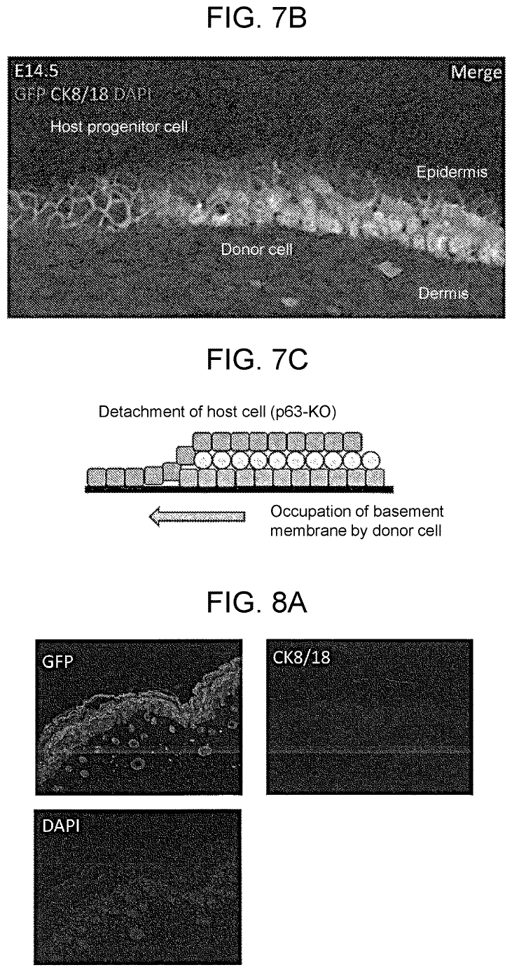

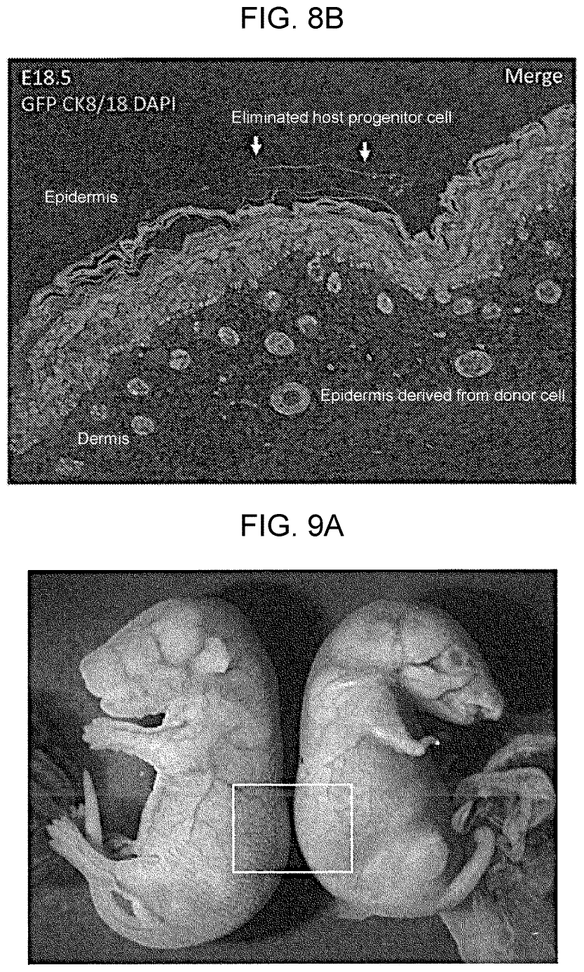

Epidermal progenitor cells (K8/K18-positive cells) are found in skin surfaces of p63-knockout mice. However, cells derived from p63 knockout were not found in the epidermis of chimeric individuals of E19.5 (see, for example, data on the keratinocytes of Table 1). Accordingly, the skin of chimeric individuals of E14.5 was observed. CK8/K18-positive host cells (derived from a p63-knockout embryo) and GFP-positive cells (derived from the donor cells) were differentially stained by fluorescent staining. Also, the expression of p63 was confirmed by fluorescent staining at the same time therewith. As a result, as shown in FIG. 7A, a skin region was found where the presence of CK8/K18-positive host cells (derived from a p63-knockout embryo) and GFP-positive cells (derived from the donor cells) was able to be confirmed on the same skin.

When these photographs were merged, as shown in FIG. 7B, GFP-positive/p63-positive donor cells were found on the basement membrane, whereas a manner was observed in which CK8/K18-positive host cells were peeled up and pushed away by the donor cells and detached from the basement membrane.

In previous organ complementation methods, donor cells occupied an empty niche ascribable to an organ defect, whereby an organ derived from the donor cells was formed (see WO2010/021390A and WO2008/102602A, which are incorporated herein by reference in their entirety). By contrast, according to the results of the present Examples, a manner was observed in which the donor cells did not occupy an empty niche and instead pushed away and removed the existing host cells. This suggested that host cells are removed by donor cells through cell competition.

GFP-positive donor cells and CK8/K18-positive host cells in the skin of chimeric individuals of E18.5 were observed under a fluorescence microscope. As a result, on E18.5, cells derived from the host were removed from the basement membrane, and the basement membrane was substantially occupied by GFP-positive donor cells.

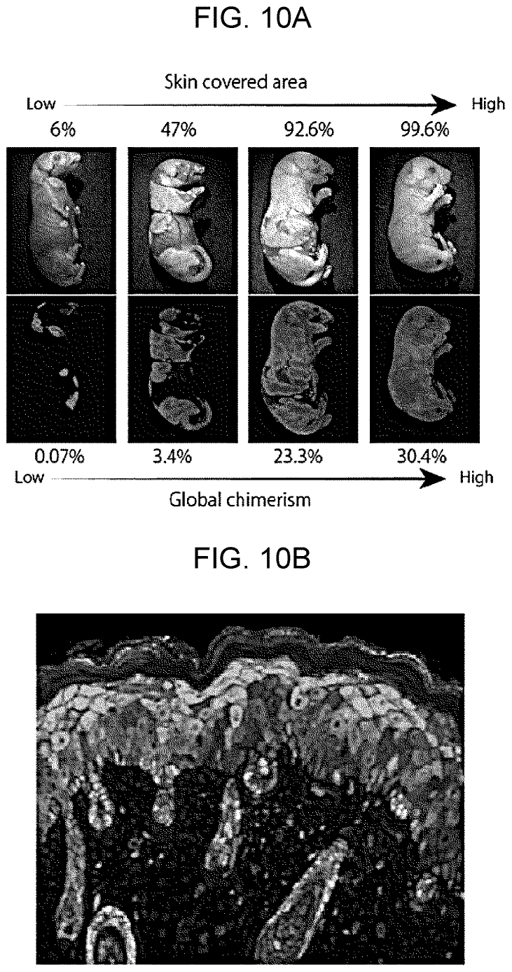

The relationship between the chimeric rate and the covered rate of the epidermis was examined in the embryos. The chimeric rate in the embryos (global chimerism) is the ratio (%) of donor cells in the embryos. The covered rate of the skin (skin covered area) is the ratio (%) of an area where the skin has been formed. As a result, as shown in FIG. 10A, the chimeric rate was approximately 30%, and the skin was formed on almost 100% of the surface. Even when the chimeric rate was approximately 23%, the skin was formed on 90% or more of the surface. This revealed that the skin is formed at a higher ratio on the surface even if a chimeric rate is low. As a result of staining tissue sections, as shown in FIG. 10B, the epidermis and its appendages were substantially occupied by EGFP-positive cells derived from pluripotent stem cells, and the dermis was a chimera derived from the donor and the embryo (i.e., the obtained skin was semi-autologous skin). It was thus found possible to regenerate semi-autologous skin.

An important thing was that the skin can be recovered from even an individual having a low covered rate of the skin and can be used as a skin grafting material. The skin grafting material comprises the epidermis and the dermis, and grafting of this grafting material to a subject presumably promotes engraftment to the skin in the subject. By contrast, when the epidermis is contaminated with host cells, the skin may be rejected as mentioned later and engraftment of grafted skin may be impaired.

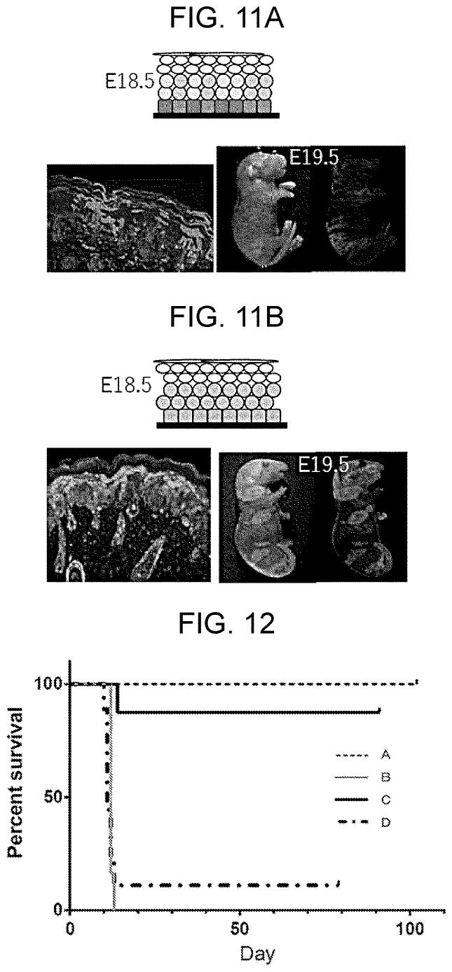

As a result of actually constructing a chimeric embryo as described above using a wild-type embryo instead of the p63-KO embryo and donor cells, as shown in FIG. 11A, the donor cells and embryo-derived host cells coexisted in the epidermis. The epidermis was contaminated with the host cells at a large ratio. When the obtained skin (comprising the epidermis and the dermis) was grafted to another individual, this skin was rejected. By contrast, as a result of actually constructing a chimeric embryo as described above using the p63-KO embryo and donor cells, as shown in FIG. 11B, almost 100% or 100% of the epidermis was derived from the donor cells. When the skin (comprising the epidermis and the dermis) of a region where the epidermis was formed was grafted to another individual, this skin was engrafted in this individual. The skin having the epidermis, almost 100% or 100% of which was derived from the donor cells, was easy to collect from the chimera constructed using the p63-KO embryo and the donor cells. Furthermore, the skin having the epidermis, almost 100% or 100% of which was derived from the donor cells, was able to be suitably used in skin grafting.

An experiment was further conducted to graft, onto B6 lineage, the epidermis and the dermis of the skin (the skin of the present disclosure) constructed as described above using a p63-KO embryo (BDF1 lineage) expressing EGFP and donor cells (B6 lineage) (C group). For controls, experiments were conducted on autografting (grafting of the skin of BDF1 lineage expressing EGFP to BDF1 lineage) as an A group and heterologous skin grafting (grafting of the skin of BDF1 lineage expressing EGFP to the skin of B6 lineage) as a B group and a D group. In the A group, the skin collected from a BDF1-Tg (CAG-EGFP) mouse was grafted to another BDF1-Tg (CAG-EGFP) mouse. The BDF1-Tg (CAG-EGFP) mouse was F1 of an EGFP-expressing B6 mouse and a DBA2 mouse. In the A group, both the epidermis and the dermis of the graft immunologically matched the graft recipient. In the B group, the skin collected from a BDF1-Tg (CAG-EGFP) mouse was grafted to a B6-Tg (CAG-EGFP) mouse. In the B group, neither the epidermis nor the dermis of the graft immunologically matched the graft recipient. In the D group, a chimeric mouse was prepared by injecting wild-type B6-derived ES cells to a fertilized embryo of a BDF1-Tg (CAG-EGFP) mouse expressing EGFP and having wild-type p63 gene, and a chimeric skin graft (having a chimeric epidermis of the donor and the host embryo) collected from the chimera was grafted to a B6-Tg (CAG-EGFP) mouse. In the D group, both the epidermis and the dermis of the graft comprise a host embryo-derived component that does not immunologically match the graft recipient.

In the experiments described above, the graft survival rate of the skin thus grafted was observed. The results were as shown in FIG. 12. As shown in FIG. 12, in the A group, 100% of the autografted skin was engrafted. By contrast, in the B group and the D group, the engraftment of the heterologously grafted skin was drastically deteriorated within 2 weeks. The skin of the present disclosure (C group) exhibited favorable engraftment over a long period. As is evident from these results, provided that the epidermis is constituted by autologous cells, high engraftment is shown even when the dermis is constituted by autologous and heterologous cells. In the skin of the present disclosure, the epidermis, follicles, sebaceous glands, and sweat glands were derived from the donor cells, and the dermis, nerves, blood vessels, and pilomotor muscles were chimerically derived from the donor cells and the embryonic cells. These results suggested that immunologically incompatible cells contained in the dermis, nerves, blood vessels, and pilomotor muscles rarely influence long-term engraftment of grafted skin.

Instead of the experiment of the C group, an experiment was conducted to graft, to B6 lineage, the epidermis and the dermis of the skin (the skin of the present disclosure) constructed as described above using a p63-KO embryo (BDF1 lineage) and donor cells (B6 lineage) expressing EGFP (C′ group). In this case, the engraftment of the EGFP-expressing donor cells was partially rejected. The EGFP expression induced rejection, suggesting that donor cells are preferably free of an antigen (particularly, a foreign or xenogeneic antigen).

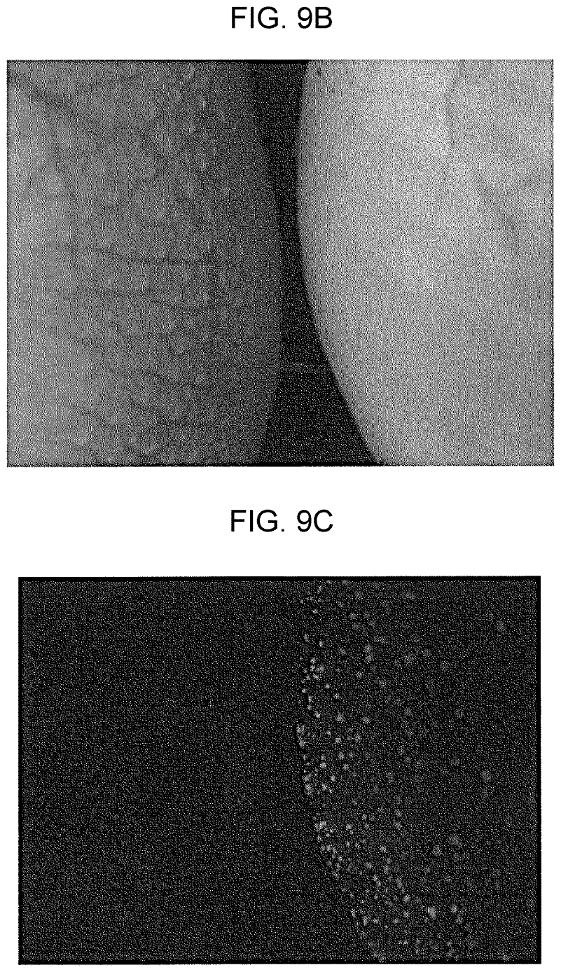

It has been reported in previous research that cells are engrafted to an embryo by the transuterine injection of the cells to the embryo (see Cohen et al., PNAS, 113 (6): 1570-5, 2016, which is incorporated herein by reference in its entirety). Accordingly, in the present Examples, HaCaT cells, immortalized human keratinocytes, were labeled with EGFP and then grafted to a p63-knockout embryo of E13.5 by injection into the amniotic cavity. No immunosuppressant was used for grafting. On E16.5, the embryo was isolated, and GFP signals derived from the donor cells were observed under a stereomicroscope and under a fluorescence microscope. As a result, as shown in FIGS. 9A and 9B, the HaCaT cells contributed to the skin. In addition, as shown in FIG. 9C, the engraftment of the cells to the embryo was observed.

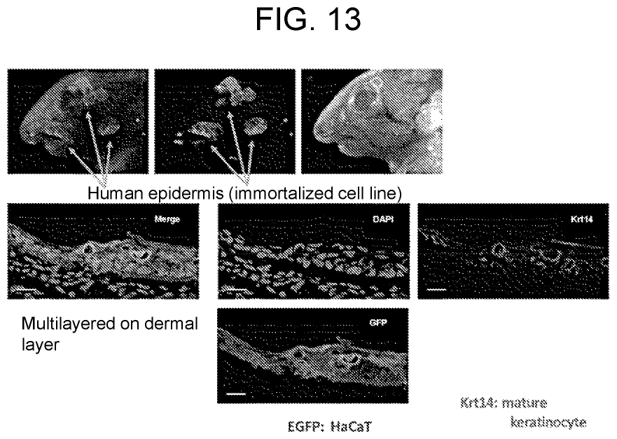

The mouse to which the HaCaT cells contributed was allowed to grow up to E18.5. As a result, as shown in FIG. 13, the epidermis derived from human epidermal cells was constructed in a partial surface of the obtained chimeric mouse. Sections of the obtained skin were prepared, and the nuclei were stained with DAPI so that a mature keratinocyte marker Krt14 was stained by immunohistological staining. When observed under a fluorescence microscope, as shown in FIG. 13, the GFP-labeled human epidermis formed a Krtl4-positive epidermis on the mouse dermis, while the epidermis had a multilayer structure found in normal skin. Thus, a skin tissue having a human epidermis was able to be constructed on the skin of a xenogeneic animal. It is thus considered possible to also construct a human epidermis on a pig dermis. The pig skin may be used as a skin grafting material for humans. It is thus expected that engraftment efficiency is enhanced by using a human epidermis (see FIG. 12).

In this way, the skin having an epidermis consisting of autologous cells can be constructed on the dermis. When the epidermis consisted of autologous cells or immunologically compatible cells, the grafted skin comprising the epidermis exhibited favorable long-term engraftment. In this respect, immunologically incompatible cells, if contained in the dermis, rarely influence the engraftment of the grafted skin. This suggests that the skin comprising an epidermis consisting of autologous cells prepared from a p63-disrupted nonhuman animal embryo and human cells, and a dermis comprising the nonhuman animal cells is capable of being preferably used in, for example, autologous skin grafting to humans.

1. An isolated skin tissue comprising an epidermis, a basement membrane, and a dermis, wherein the epidermis is attached to one surface of the basement membrane, and the dermis is attached to the other surface of the basement membrane; and the epidermis comprises first cells, and the dermis comprises second cells, wherein the second cells have low or no ability to differentiate into mature epidermal cells.

2. The skin tissue according to claim 1, wherein second individual's cells are cells having loss of function in p63.

3. The skin tissue according to claim 1 or 2, wherein the epidermis is a mature epidermis.

4. The skin tissue according to any one of claims 1 to 3, wherein 95% or more of cells of the epidermis are the first cells, and 75% or more of cells of the dermis are the second cells.

5. A method for preparing a skin tissue, comprising:
providing a host embryo incapable of autonomously developing a mature epidermis;
injecting donor cells having ability to differentiate into a mature epidermis to the host embryo, wherein the injection is performed under conditions suitable for engraftment of the cells to the skin;
allowing the obtained embryo to grow in the uterus of a pseudo-pregnant surrogate mother mammal by grafting to the uterus thereof to obtain an individual having a mature epidermis from the mammal; and
isolating a skin tissue comprising a mature epidermis comprising cells derived from the donor cells, a basement membrane, and a dermis comprising cells derived from the host from the obtained individual.

6. The method according to claim 5, wherein the host embryo incapable of developing a mature epidermis is a p63-knockout embryo.

7. The method according to claim 5 or 6, wherein the donor cells having ability to differentiate into a mature epidermis are cells selected from the group consisting of pluripotent cells and cells destined for the epidermis.

8. The method according to any one of claims 5 to 7, wherein the embryo is an embryo at any stage of development from an eight-cell stage to a blastocyst stage.

9. The method according to any one of claims 5 to 7, wherein the embryo is an embryo at any stage of development from a blastocyst stage to the formation of an epidermis comprising K8/K18-positive cells.

10. The method according to any one of claims 5 to 7, wherein the embryo is an embryo having an epidermis of K8/K18-positive cells.

11. A method for preparing a nonhuman mammalian individual, comprising bringing cells having ability to form a chimera and having ability to differentiate into a mature epidermis, or cells committed to an epidermis or cells of epidermal lineage (donor cells) into contact with a body surface of a nonhuman mammalian individual (host) before formation of epidermal basal cells, thereby obtaining a nonhuman mammalian individual having a skin tissue comprising cells of the host and the donor cells.
